(19) United States
(12) Patent Application Publication
(10) Pub. No.: US 2004/0219266 A1
(43) Pub. Date: Nov. 4, 2004

(54) Determination of homogenization efficiency based on light transmittance

(76) Inventors: Adam S. Milliken, Columbus, OH (US); Chron-Si Lai, Blacklick, OH (US)

Correspondence Address: Ross Products Division of Abbott Laboratories, 625 Cleveland Avenue, Columbus, OH, 43221, US

(21) Appl. No.: 10/427,018

(22) Filed: Apr. 30, 2003

(51) Int. Cl. G01N 33/02

(52) U.S. Cl. 426/231

(57) ABSTRACT

Disclosed is a method for the determination of homogenization efficiency based on a measurement of light transmittance through a homogenized liquid produc, wherein homogenization efficiency is determined by in-line measurement or by benchtop light transmittance analysis.

### FIELD OF THE INVENTION

The invention relates to a method for determining homogenization efficiency based on a measurement of light transmittance through a homogenized liquid product, including in-line determination of homogeneity of nutritional or other liquid formulas in the visible and near infrared light range.

### BACKGROUND OF THE INVENTION

There are many different commercial products that come in the form of homogenized liquids, including nutritional liquids such as infant formula, adult nutrition products, and so forth. Most of these products are in the form of homogenized emulsions stabilized by the colloidal nature of the suspended particles.

As background, colloidal systems comprise small, uniformly-sized particles (gaseous, liquid or solid) as a discontinuous phase evenly suspended in a continuous gaseous, liquid or solid phase, wherein the particles are so small that they become self-suspending and unaffected by the forces of gravity. Non-limiting examples of colloidal systems can include aerosols (solid particles in a gas), emulsions (solid or liquid particulates in liquid), or even foams (gases suspended in a liquid).

Homogenized milk is a common example of a colloidal system. In this type of system, cows milk or other naturally sourced milk is subjected to homogenization, a mechanical process that reduces the size of the fat particles found in milk from abouit 6-10 microns to about 1-2 microns, so that the small suspended fat particles form a colloidal system which is then stable and also unaffected by gravity.

Homogenization processes are well known in the various formulation and manufacturing arts, and are generally needed or at least desirable to provide prolonged shelf-life of an emulsified liquid or other similar product. Homogenization processes are well known in many different manufacturing systems, most of which involve the stabilization of an emulsion system by reducing the size of suspended particles so that the very small particles, i.e., colloidal particles, will not settle out of the suspension by the forces of gravity. Moreover, not only can homogenization processes affect the physical stability and shelf life of an emulsion system, it can also have a substantial affect on product characteristics such as taste, color, light sensititivity, and foaming tendencies.

Examples of commercial products that greatly depend upon homogenization processes and the efficiency of those processes include nutritional liquids such as infant formulas, adult nutritional liquids, and other similar products that require homogenization of fat or lipid particles in an emulsion system.

The homogenization efficiency of a manufacturing method relates to the extent to which fat particles are uniformly reduced to the desired colloidaol size, and can be determined during various phases of plant processing by a variety of methods. If differences in the extent of homogenization of particles are noted from sample to sample with a single batch or from batch to batch, such differences may be indicative of system upsets such as the introduction of entrained air, homogenization valve wear or loss of homogenization pressure. If the extent of homogenization of the fat or other particles is not at an acceptable level, entire batches may be formulated or manufactured that result in unacceptable product that must then be discarded. Early detection of such system upsets is desirable so that repairs or maintenance of the system can be performed and the expense and loss of time for reprocessing can be minimized.

Several methods for determining the extent to which particles are efficiently homogenized in an emulsion system are known, non-limiting examples of which include, direct microscopic examination of a sample of the emulsion, spectrophotometric turbidity methods, and use of a Coulter Counter to count and size fat or other particles present in a sample.

Direct microscopic examination of a sample emulsion has been done by first adding ammonium hydroxide in a concentration of 5 N to solubilize protein and reduce turbidity. The sample is then diluted with 40% glycerin to slow Brownian movement so that counting of the fat particles is made easier. The particles are counted and their size estimated for 5 fields. Size estimations are made by comparison to a calibrated micrometer in the eyepiece. Homogenization has been considered to be acceptable where 80% of the particles are 2 i or smaller.

Direct microscopic evaluation of the extent of homogenization has several drawbacks. These drawbacks include dependency on the accuracy of the dilution in the process, variation in depth of the sample well to be observed may skew results, the area of the slide chosen to view fields is arbitrary and may not be representative of the entire sample, and careful use by experience operators is required. Only a rough size of fat globules in an emulsion can be determined and only gross homogenization errors can be detected.

Spectrophotometric methods have been used to correlate the turbidity of an emulsified sample to the extent of homogenization of the sample. Similar to the direct microscopic method, 5 N ammonium hydroxide is added to remove turbidity due to the presence of protein. Samples are then diluted with distilled water and the optical density of the sample is determined by use of a spectrophotometer at a wavelength of 999 i. The spectrophotometer value is then corrected by multiplying the known fat percentage of the emulsion or mixture.

The Coulter Counter test involves the use of an instrument which determines both the number and size of particles. A sample to be tested is diluted with saline and placed in a beaker in which an aperture tube with a precision micro aperture provides the only path for the conduction of electricity. A mercury manometer metering system draws a measured volume of diluted sample through the aperture tube. Each particle displaces the electrically conductive saline solution, momentarily changing the resistance between the electrodes and producing a voltage pulse which is proportional to the particle volume. The electric pulses are amplified and fed to a threshold circuit having an adjustable threshold level. If this level is reached or exceeded by a pulse, the pulse is counted.

Unfortunately, the Coulter Counter is an extremely sensitive instrument that is subject to both acoustical and electrical interferences. The saline solution must be filtered prior to use in the dilution of samples and all glassware must be rinsed repeatedly with the filtered saline to eliminate dust from its surface. Also, because the aperture is small, it is frequently plugged by large particles, especially dust, and may require the operator to monitor the aperture through a low power microscope. When attempting to access fat droplets in a liquid formulation, the Coulter Counter readings can also be unduely influenced by insoluble particles other than the fat droplets, thus impacting the reliability of the resulting reading.

The methods described above are all laboratory methods that require transportation time and dilution with analytical reagents. The requirement of transporting samples and involved laboratory analysis extends the time required to diagnose an upset in the homogenization process and to correct the upset. Further, the addition of ammonium hydroxide to the samples will break up flocculated particles that have formed in the sample and the flocs will not be detected.

There is a need for a quick method of in-line determination of homogenization efficiency. There is further a need for a method of determining homogenization efficiency that does not require dilution of the sample.

The invention provides an in-line method for the determination of homogenization efficiency by which dilution of the sample is not required.

### SUMMARY OF THE INVENTION

One embodiment of the invention provides a method for determining the homogenization efficiency of a liquid having particles suspended therein. The method comprises the steps of obtaining a sample of an homogenized liquid; passing light through the sample; measuring an amount of light that is transmitted through the sample; and comparing the amount of light transmitted through the sample to a calibration curve of known homogenization efficiencies.

In a further embodiment, the sample of homogenized liquid is an emulsion and the particles are fat globules.

In a further embodiment, the sample of homogenized liquid is taken in-line in a production process.

In a further embodiment, the light transmitted through the sample has a wavelength of between about 400 and about 1100 nm.

In still a further embodiment, the light transmitted through the sample has a wavelength of between about 500 and about 1500 nm.

In another embodiment, the invention is a method for in-line determination of homogenization efficiency of an emulsion, the method comprising the steps of positioning a sanitary in-line light absorption sensor; aligning a light source to direct light waves in the direction of the light absorption sensor; orienting a sample of an homogenized emulsion between the light source and the light absorption sensor; measuring an amount of light transmitted through the sample; and comparing the measured amount of light to a calibration curve of known homogenization efficiencies.

In a further embodiment, the light transmitted through the sample has a wavelength of between about 400 and about 1100 nm, and in still a further embodiment, the light transmitted through the sample has a wavelength of between about 500 and about 1500 nm.

In another embodiment, the invention provides a method for determination of homogenization efficiency of an emulsion, the method comprising the steps of positioning a sanitary light absorption sensor; aligning a light source to direct light waves in the direction of the light absorption sensor; orienting a sample of an homogenized emulsion between the light source and the light absorption sensor; measuring an amount of light transmitted through the sample; and comparing the measured amount of light to a calibration curve of known homogenization efficiencies.

In a further embodiment, the method further comprises the step of diluting the sample prior to orienting the sample between the light source and the light absorption sensor.

### BRIEF DESCRIPTION OF THE DRAWINGS

FIG. 1 is a graph depicting the relationship between the output from an in-line light absorption sensor at a constant total solids value versus varying homogenization pressures.

FIG. 2 is a graph depicting the relationship between the output from an in-line light absorption sensor at 2000 and 4000 psi versus percent total solids.

FIGS. 3 to 7 are graphs depicting the relationship between transmittance and homogenization pressure for several products tested using a benchtop spectrophotometer.

### DETAILED DESCRIPTION OF THE INVENTION

Homogenization of gas, liquid or solid particles in liquid and, in particular, the homogenization of fat globules in a liquid suspension to form an emulsion, is a common process in the production of consumer products. Examples of consumer products that commonly undergo homogenization during production include milk products; ready-to-serve infant formulas and dietary supplements for infants, children and adults; and personal hygiene and beauty products such as lotions and cosmetics.

The ability to detect shifts in the day-to-day, batch-to-batch or within batch homogenization efficiencies in putative consumer products facilitates quick and easy adjustments in processing operations. There is particular value in the detection of homogenization efficiencies of putative products if the measurement of homogenization can be taken while the product remains in-line, i.e., without having to remove the product from the production line, in order for the measurement to be taken. Determination of homogenization efficiency according to the invention may be incorporated into a production plant or may be used as a laboratory bench method.

For the purposes of this invention, the term “homogenization” means the mechanical reduction of a particle to a uniform size within a suspension. The term “homogenization efficiency” is a value directly proportional to the transmittance of light at a set wave length over the range of 400 to 1500 nm that is passed through a sample of a homogenized sample. Homogenization efficiency may also refer to a measurement of the extent to which particles have been uniformly sized and remain suspended in a colloidal system. Homogenization efficiency is effected by factors such as homogenization pressure, particle size, range of particle size and the degree of agglomeration of particles.

The term “homogenized sample” means a solid or liquid sample having gas, solid or liquids of reduced size suspended in the solid or liquid to form a colloidal system.

The term “colloidal system” means a suspension of gaseous, liquid, fat or solid particles in gases, solids or liquids, including aerosols, emulsions and foams.

The term “light transmittance” means the amount or percentage of light intensity that passes through a sample. Light transmittance measurements can be made by either diluted samples of products on benchtop spectrophotometers or non-dilute samples of products using a sanitary in-line light absorption sensor.

The invention uses light transmittance in the visible and near-infrared range (400 to 1100 nm) for a quick determination of homogenization efficiency. In theory, light will be either absorbed or scattered by particles larger than its wavelength. Homogenized emulsions generally produce a majority of fat droplets of a size between 0.5 and 1.5 μm. Therefore, if light of a set wavelength, or light of a range of wavelengths between 500 and 1500 nm, is passed through a homogenized sample of an emulsion, the transmittance will be inversely proportional to the fat particle size and directly proportional to the homogenization efficiency.

The method described herein allows the detection of small shifts in homogenization efficiency from batch to batch and within an individual batch. The present invention has shown that transmittance testing can be done without solubilizing the protein, and with or without dilution. When in-line light absorption sensors are used, no sample preparation is required. When benchtop spectrophotometers are used, preparation requires a simple dilution. Therefore, the measurements are quick and easy.

The method of the present invention has been shown to correlate with homogenization efficiency for a product containing insoluble fibers up to and over 40 microns in size. The proposed method was shown to correlate with homogenization efficiency using dilute samples on a benchtop spectrophotometer. It was then determined that the same correlation could be achieved using non-dilute samples on an in-line absorption sensor. Therefore, readings can be taken continuously in-line or analytically with minimal sample preparation.

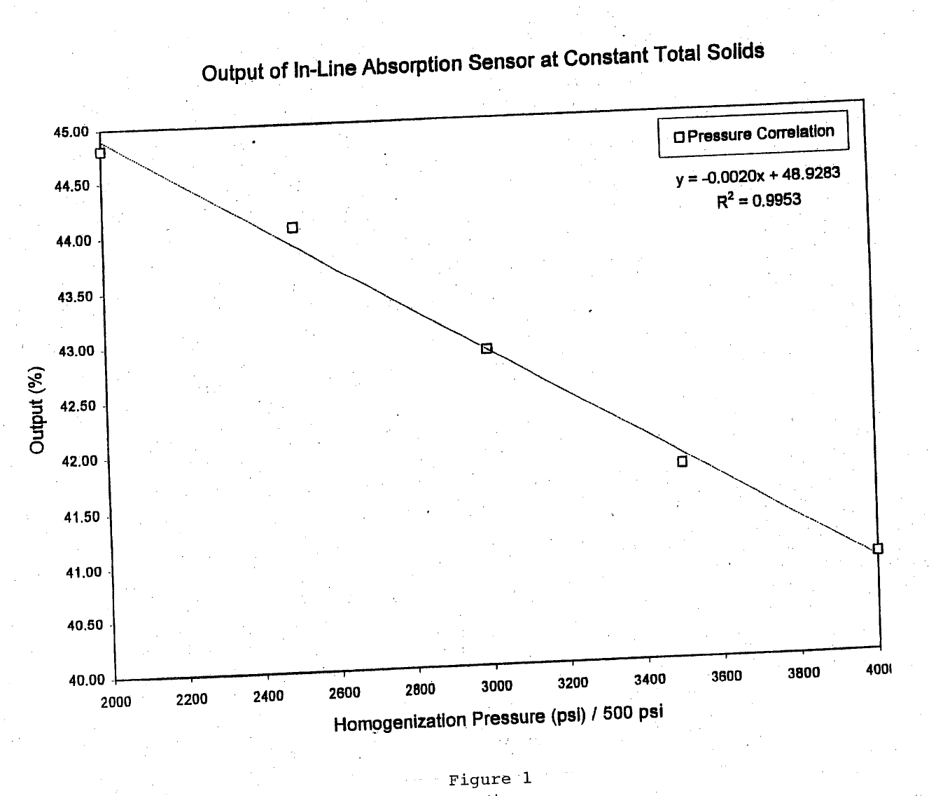

FIG. 1 shows the output of an in-line light absorption sensor as a function of homogenization pressure in which 0% output=2 CU and 100% output=7 CU where CU is defined as a reduction in the light intensity by 90%. Therefore, 1 CU is equivalent to a transmittance of 10% and 2 CU is equivalent to a transmittance of 1%. As can be seen, the amount of light detected at the sensor vs. the amount of light emitted by the lamp is very small for these samples. It is known that homogenization pressure directly effects homogenization efficiency, with higher pressures producing higher efficiencies. The sensor output is linear with homogenization pressure. Higher pressure gives less absorption/greater transmittance. The data was collected using a Concentration Monitor Model 116/AF marketed by Optik-Dannlat GmbH of Essen, Germany. The emulsion tested was a nutritional liquid with suspended fat particles which contains approximately 3.3% fat, 3.8% protein and 23% total solids (JEVITY FOS). The insoluble fibers in this product are generally up to and over 40 microns. The sample was emulsified at 900 to 1100 psig, heated to 291-295° F. for 5 seconds, cooled to 160-180° F., homogenized and then cooled to 40° F.

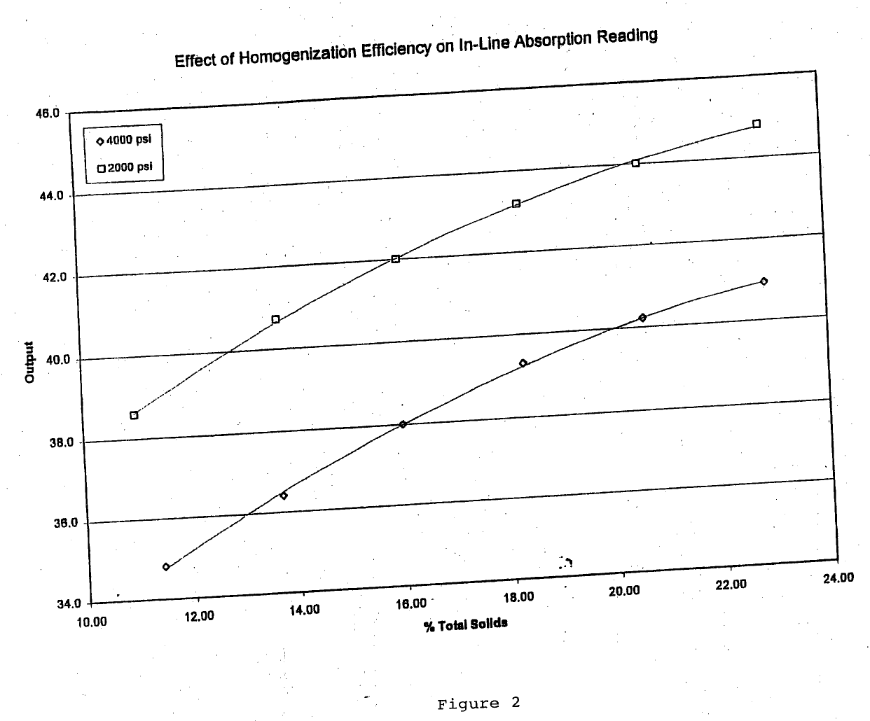

In-line absorption sensors can be used to measure total solids. FIG. 2 shows the sensitivity of a sensor to both total solids and homogenization pressure. Higher homogenization pressures will increase homogenization efficiency. Therefore, while light absorption analysis alone may not be the best test for total solids because particle size is not considered, it is an excellent test of homogenization efficiency for a given product at constant fat level calibrated against homogenization pressure.

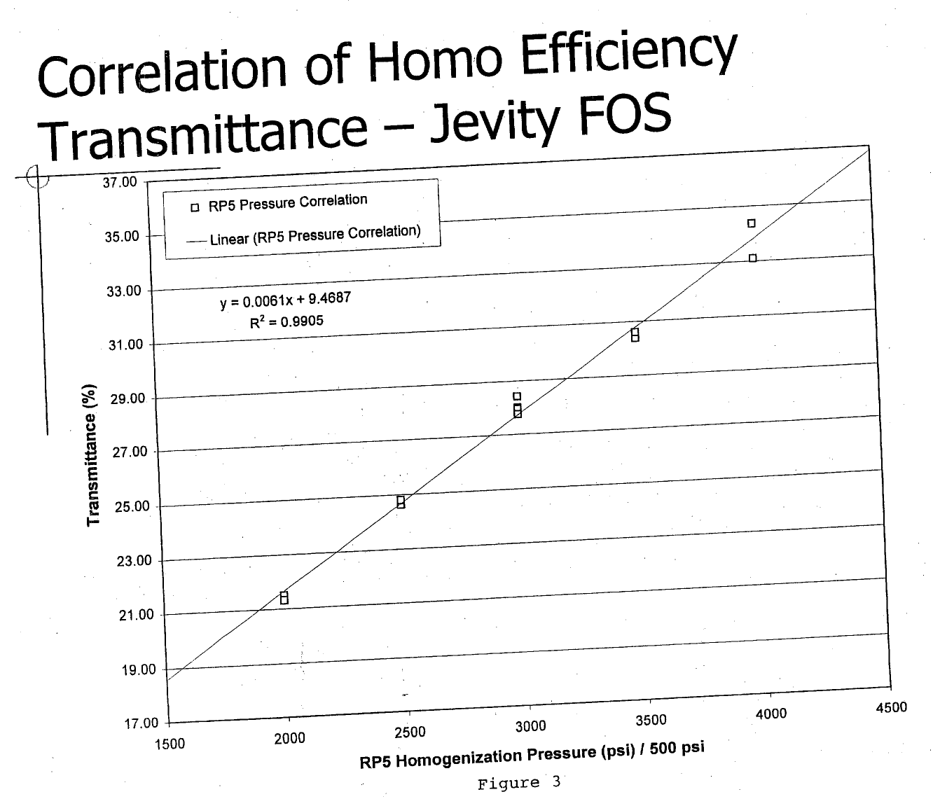

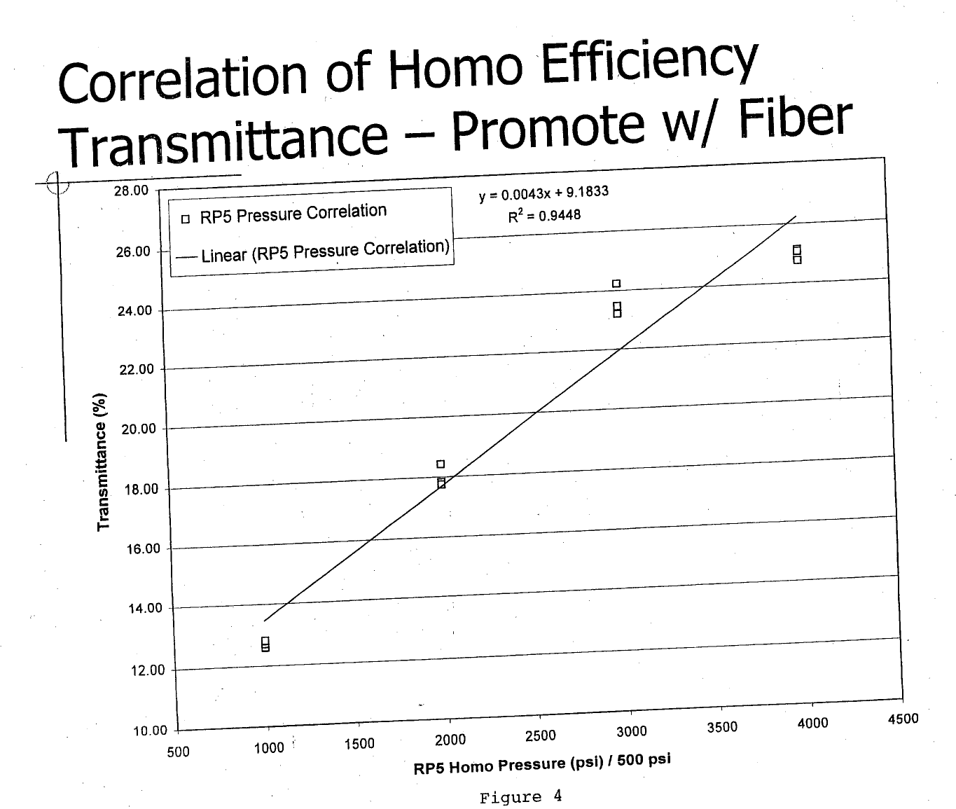

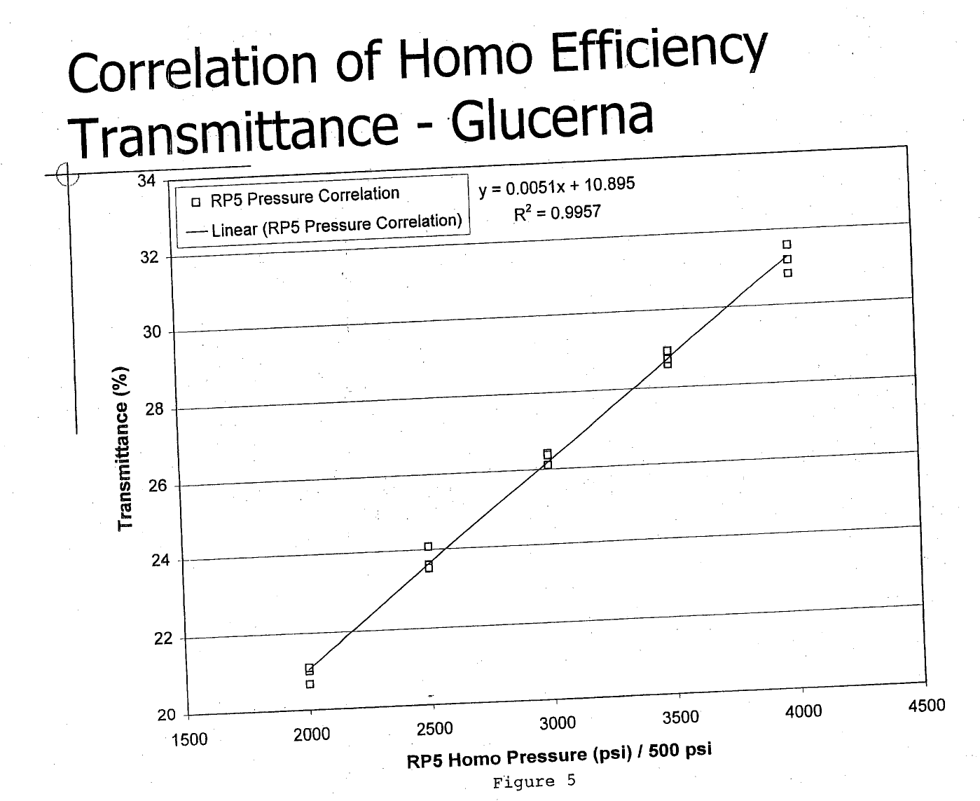

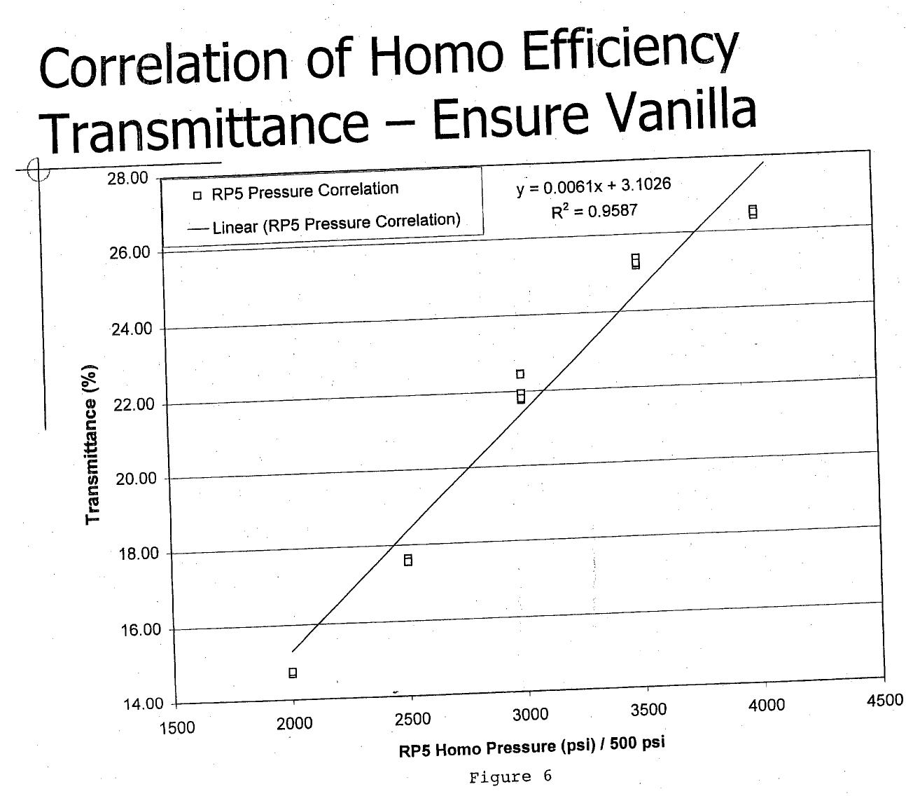

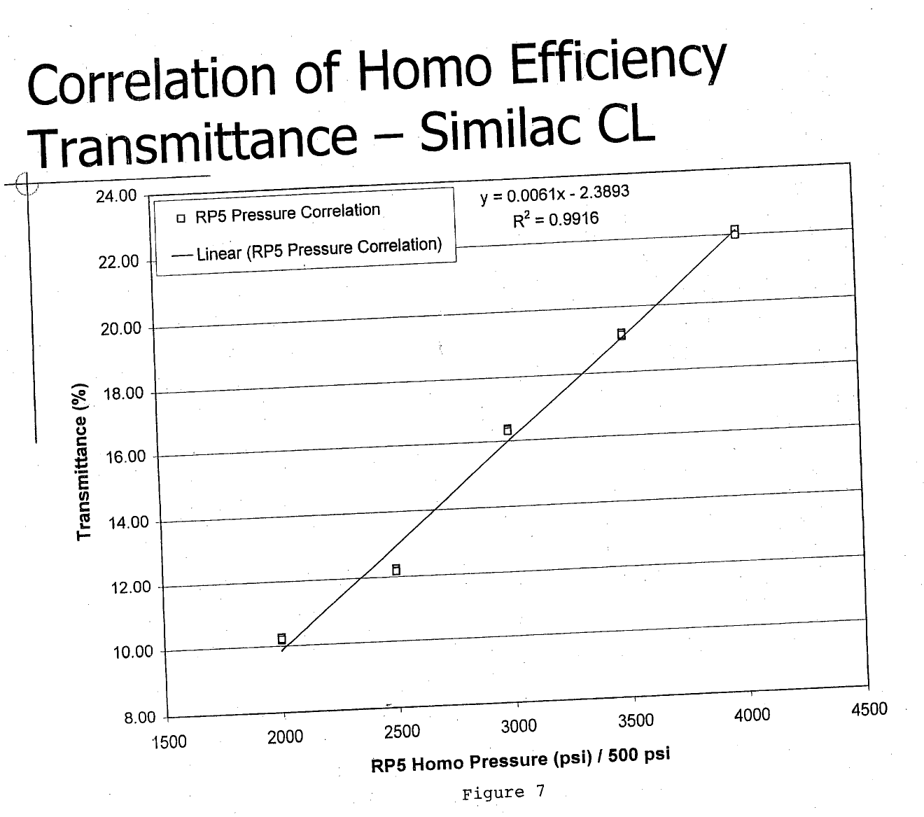

The methods described herein allow for direct measurement of homogenization efficiency of any given product/total solids combination. Therefore, one process system can be calibrated to another based on homogenization pressure by using the processes. A drop in homogenization efficiency on a single process system (due to entrained air, homogenization valve wear, loss of homogenization pressure, or loss of homogenization feed pressure) can also be detected using this method. A standard light transmittance vs. total solids calibration may be developed for each product for measurements to be taken during production. During production, the transmittance would be recorded in-line, or analytically using a spectrophotometer such as a Shimadzu UV-1601 spectrophotometer, and compared to the light transmittance vs. total solids curve. FIGS. 3 to 7 illustrate the results for several different diluted products tested at different homogenization pressures on the benchtop spectrophotometer. A similar linear correlation is observed using both the benchtop spectrophotometer and the concentration monitor. If the measurement of transmittance is significantly low or high, it would be an indication of poor homogenization, and suggest the need for reprocessing.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While some potential advantages and objects have been expressly identified herein, it should be understood that some embodiments of the invention may not provide all, or any, of the expressly identified advantages and objects.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for determining the homogenization efficiency of a liquid having particles suspended therein, the method comprising the steps of:
obtaining a sample of a homogenized liquid;
passing light through the sample;
measuring an amount of light that is transmitted through the sample; and
comparing the amount of light transmitted through the sample to a calibration curve of known homogenization efficiencies.

2. The method of claim 1 wherein the sample of homogenized liquid is an emulsion and the particles are fat globules.

3. The method of claim 1 wherein the sample of homogenized liquid is taken in-line in a production process.

4. The method of claim 1 wherein the light transmitted through the sample has a wavelength of between about 400 and about 1100 nm.

5. The method of claim 1 wherein the light transmitted through the sample has a wavelength of between about 500 and about 1500 nm.

6. A method for in-line determination of homogenization efficiency of an emulsion, the method comprising the steps of:
positioning a sanitary in-line light absorption sensor;
aligning a light source to direct light waves in the direction of the light absorption sensor;
orienting a sample of a homogenized emulsion between the light source and the light absorption sensor;
measuring an amount of light transmitted through the sample; and
comparing the measured amount of light to a calibration curve of known homogenization efficiencies.

7. The method of claim 6 wherein the light transmitted through the sample has a wavelength of between about 400 and about 1100 nm.

8. The method of claim 6 wherein the light transmitted through the sample has a wavelength of between about 500 and about 1500 nm.

9. A method for determination of homogenization efficiency of an emulsion, the method comprising the steps of:
positioning a sanitary light absorption sensor;
aligning a light source to direct light waves in the direction of the light absorption sensor;
orienting a sample of a homogenized emulsion between the light source and the light absorption sensor;
measuring an amount of light transmitted through the sample; and
comparing the measured amount of light to a calibration curve of known homogenization efficiencies.

10. The method of claim 9 further comprising the step of diluting the sample prior to orienting the sample between the light source and the light absorption sensor.
